(19) United States
(12) Patent Application Publication
(10) Pub. No.: US 2010/0076057 A1
(43) Pub. Date: Mar. 25, 2010

(54) TARGET DNA INTERFERENCE WITH crRNA

(75) Inventors: Erik J. Sontheimer, Kenilworth, IL (US); Luciano A. Marraffini, Chicago, IL (US)

Correspondence Address: Casimir Jones, S.C., 2275 DEMING WAY, SUITE 310, MIDDLETON, WI, 53562, US

(73) Assignee: Northwestern University, Evanston, IL (US)

(21) Appl. No.: 12/565,589

(22) Filed: Sep. 23, 2009

Related U.S. Application Data
(60) Provisional application No. 61/099,317, filed on Sep. 23, 2008.

(51) Int. Cl. A61K 31/7088; A61P 31/00

(52) U.S. Cl. 514/44 A

(57) ABSTRACT

The present invention provides methods, systems, and compositions for interfering with the function and/or presence of a target DNA sequence in a eukaryotic cell (e.g., located in vitro or in a subject) using crRNA and CRISPR-associated (cas) proteins or cas encoding nucleic acids. The present invention also relates to a method for interfering with horizontal gene transfer based on the use of clustered, regularly interspaced short palindromic repeat (CRISPR) sequences.

The present application claims priority to U.S. Provisional Application Ser. No. 61/099,317, filed Sep. 23, 2008, which is herein incorporated by reference in its entirety.

This invention was made with government support under grant number 1 R01 GM072830 awarded by the National Institutes of Health (NIGMS) and grant number 1 R03 AI079722 awarded by the National Institutes of Health (NIAID). The government has certain rights in the invention.

### FIELD OF THE INVENTION

The present invention provides methods, systems, and compositions for interfering with the function and/or presence of a target DNA sequence in a eukaryotic cell (e.g., located in vitro or in a subject) using crRNA and CRISPR-associated (cas) proteins or cas encoding nucleic acids. The present invention also relates to a method for interfering with horizontal gene transfer based on the use of clustered, regularly interspaced short palindromic repeat (CRISPR) sequences.

### BACKGROUND OF THE INVENTION

The horizontal transfer of genetic material has played an important role in bacterial evolution (Thomas and Nielsen, Nat. Rev. Microbiol. 3, 711 (2005), herein incorporated by reference in its entirety) and also supports the spread of antibiotic resistance among bacterial pathogens (Furuya and Lowy, Nat. Rev. Microbiol. 4, 36 (2006), herein incorporated by reference in its entirety). The rise of hospital- and community-acquired meticillin- and vancomycin-resistant S. aureus (MRSA and VRSA, respectively) is directly linked to the horizontal transfer of antibiotic resistance genes by plasmid conjugation (Weigel et al., Science 302, 1569 (2003), herein incorporated by reference in its entirety) and has made treatment and control of staphylococcal infections increasingly difficult (Stevens, Curr. Opin. Infect. Dis. 16, 189 (2003), herein incorporated by reference in its entirety). Understanding the limitations that are placed on HGT has therefore become an important research goal.

Clustered, regularly interspaced short palindromic repeat (CRISPR) sequences are present in ˜40% of eubacterial genomes and nearly all archaeal genomes sequenced to date, and consist of short (˜24-48 nucleotide) direct repeats separated by similarly sized, unique spacers. They are generally flanked by a set of CRISPR-associated (cas) protein-coding genes that are important for CRISPR maintenance and function. In Streptococcus thermophilus and Escherichia coli, CRISPR/cas loci have been demonstrated to confer immunity against bacteriophage infection by an interference mechanism that relies on the strict identity between CRISPR spacers and phage target sequences. What is needed are ways to regulate gene transfer to gain better control and regulation of biological processes associated with horizontal transfer of genetic material.

### SUMMARY OF THE INVENTION

The present invention provides methods, systems, and compositions for interfering with the function and/or presence of a target DNA sequence in a eukaryotic cell (e.g., located in vitro or in a subject) using crRNA and CRISPR-associated (cas) proteins or cas encoding nucleic acids. The present invention also relates to a method for interfering with horizontal gene transfer based on the use of clustered, regularly interspaced short palindromic repeat (CRISPR) sequences.

In some embodiments, the present invention provides methods of inhibiting the function and/or presence of a DNA target sequence in a cell (e.g., eukaryotic cells) comprising: administering crRNA and one or more cas proteins, or nucleic acid sequences encoding the one or more cas proteins, to a cell comprising a target DNA sequence, wherein the crRNA hybridizes with the target DNA sequence thereby interfering with the function and/or presence of the target DNA sequence. In certain embodiments, the administering is with a physiolocally tolerable buffer. In particular embodiments, the interfering with the function and/or presence of the target DNA sequence interference with transcription of the target DNA sequence.

In certain embodiments, the present invention provides compositions or systems comprising: i) isolated crRNA sequences; ii) one or more isolated cas proteins, or isolated nucleic acid sequences encoding the one or more cas proteins; and iii) a transfection reagent (e.g., liposomes, buffers, etc.) configured to aid in importing the crRNA into a target cell.

In some embodiments, the one or more cas proteins comprises Cas3. In other embodiments, the one or more cas proteins comprise Cas3 and Cse1-5 proteins. In further embodiments, the interfering with the function and/or presence of the target DNA sequence silences expression of the target DNA sequence. In particular embodiments, the cell is located in vitro or in a subject. In additional embodiments, the target DNA sequence is a detrimental allele that causes the subject to have a disease or condition. In other embodiments, the target DNA sequence is located within the genome of the cell. In further embodiments, the target DNA sequence is located within close proximity to a CRISPR motif sequence. In other embodiments, the cell is eukaryotic or prokaryotic. In some embodiments, the methods further comprise studying the effect on the of interfering with the function and/or presence of the target DNA sequence compared to a control cell (e.g., where both cells are eukaryotic cells).

In certain embodiments, the present invention provides methods of treating an infection comprising: administering crRNA and one or more cas proteins, or nucleic acid sequences encoding the one or more cas proteins, to a subject infected by a pathogen, wherein the crRNA hybridizes to a target DNA sequence from the pathogen thereby interfering with the function and/or presence of the target DNA sequence.

In some embodiments, crRNA sequences are obtained from public databases, such as the one at “http:” followed by “//crispr.u-psud.fr/crispr,” and described in Grissa et al., BMC Bioinformatics. 2007 May 23; 8:172 (herein incorporated by reference).

In additional embodiments, the interfering with the function and/or presence of the target DNA sequence is fatal to the pathogen. In further embodiments, the pathogen is selected from a bacteria, virus, and fungus. In other embodiments, the target DNA sequence is located within the genome of the pathogen.

In some embodiments, the present invention provides compositions and methods for regulating gene transfer, comprising: inhibiting horizontal gene transfer, wherein clustered, regularly interspaced short palindromic repeat (CRISPR) loci and CRISPR-associated (cas) protein-coding genes are configured within the DNA of a cell, tissue, or subject to inhibit horizontal gene transfer into said DNA of said cell, tissue, or subject. In some embodiments, the subject is an archeabacteria or eubacteria (e.g. Staphylococcus epidermidis). In some embodiments, horizontal gene transfer includes, but is not limited to, plasmid conjugation, phage trandsduction, DNA transformation, or the like. In some embodiments, a CRISPR loci comprises 24-48 nucleotide direct repeats of DNA sequence separated by 20-50 nucleotide unique spacers. In some embodiments, CRISPR-associated (cas) proteins comprise proteins that are important for CRISPR maintenance and function.

In some embodiments, the present invention provides a method of inhibiting horizontal gene transfer comprising providing one or more crRNA and one or more cas proteins, wherein the crRNA and one or more cas proteins are configured to inhibit horizontal gene transfer. In some embodiments, the crRNA are expressed from one or more clustered, regularly interspaced short palindromic repeat (CRISPR) loci. In some embodiments, the one or more cas proteins are expressed from one or more CRISPR-associated (cas) protein genes. In some embodiments, the crRNA and one or more cas proteins are expressed in a cell, tissue, or subject. In some embodiments, a cell, tissue, or subject is prokaryotic. In some embodiments, a cell, tissue, or subject is eukaryotic. In some embodiments, a cell, tissue, or subject is human. In some embodiments, horizontal gene transfer comprises plasmid conjugation. In some embodiments, horizontal gene transfer comprises phage trandsduction. In some embodiments, horizontal gene transfer comprises DNA transformation. In some embodiments, the CRISPR loci comprise 20-50 nucleotide direct repeats of DNA sequence separated by 20-50 nucleotide unique spacers. In some embodiments, CRISPR-associated (cas) proteins comprise proteins that are important for CRISPR maintenance and function.

### BRIEF DESCRIPTION OF THE DRAWINGS

The foregoing summary and detailed description is better understood when read in conjunction with the accompanying drawings which are included by way of example and not by way of limitation.

FIG. 1 shows a CRISPR locus in S. epidermidis. (A) Genetic organization of the S. epidermidis RP62a CRISPR locus. (B) The staphylococcal conjugative plasmid pGO400 spc1 target sequence [pGO(wt). This sequence was altered to introduce synonymous mutations [pGO(mut), with changes that do not modify the amino acid sequence encoded by the nes gene. (C) pLM305, which contains the repeats and spacer sequences cloned into pLM6 (a vector that provides IPTG-inducible expression from the Pspac promoter), and pLM306, which includes additional upstream sequences, including the leader. (D) Conjugation was carried out by filter mating; the average cfu/ml values obtained for recipients and transconjugants are shown.

FIG. 2 shows CRISPR interference uses an intact target sequence in plasmid DNA but not mRNA. (A) Disruption of the nes target sequence with a self-splicing intron. (B) Transfer of the plasmids described above was tested using wild-type S. epidermidis RP62a and the isogenic Δcrispr mutant strain, LAM104, as recipients.

FIG. 3 shows CRISPR interference during DNA transformation. (A) Introduction of the nes target sequence into the HindIII site of the staphylococcal plasmid pC194. 480 by of pGO(wt) or pGO(mut) that contain the nes target sequence were cloned into the pC194 HindIII site to generate pLM314 and pLM317, respectively. (B) S. epidermidis RP62a and the isogenic Δcrispr mutant LAM104 were transformed with the plasmids. Transformation efficiency was calculated as cfu/μg DNA.

FIG. 4 shows CRISPR interference limits all three primary pathways of horizontal gene transfer. Phage transduction, plasmid conjugation and DNA transformation are depicted. In each case, CRISPR systems interfere with the entry or maintenance of the incoming DNA.

FIG. 5 shows (A) Sequences of repeats and spacers in the RP62a CRISPR locus. Four 37-nucleotide direct repeats (DR1-4) flank three 33- or 34-nucleotide spacer sequences (spc1-3). (B) Conjugation assays using S. epidermidis ATCC12228, a strain that lacks a CRISPR locus, as recipient. (C) pLM305-based CRISPR expression. RT-PCR was performed using P37 and P6, primers that amplify the repeat spacer region of the S. epidermidis RP62a CRISPR locus. A PCR product was observed in LAM104 cells carrying pLM305 only in the presence of IPTG, but not in cells harboring the empty plasmid pLM6. A less intense product was obtained using RP62a total RNA.

FIG. 6 shows detection of a spc1 crRNA by primer extension. (A) Arrows indicate the priming sites of the sense (S, P68 in Supplementary Table S1) and antisense (A, P69) primers within spc1 of the CRISPR locus. DR denotes a CRISPR direct repeat. (B) The antisense primer shown in (A) yields a ˜40-nt product (denoted by the filled arrowhead on the right) specifically in CRISPR-positive strains. All reactions contained a primer (“r”) complementary to 5S rRNA as an internal positive control (P67), and the 26-nt 5S rRNA extension product is indicated by the open arrowhead on the right. Lanes marked “r” contained the 5S rRNA primer only, and lanes marked “A” and “S” also contained the antisense or sense primers, respectively. A dideoxy sequencing ladder is included to the left, and the sequences of spc1 (purple box) and DR1 (unfilled box) are indicated. (C) CRISPR precursor RNA is cleaved at the base of a potential stem-loop structure in each repeat.

FIG. 7 shows splicing of an intron-containing nickase gene is used for nickase activity. (A) Disruption of the nes sequence with a self-splicing intron. A group I self-splicing intron (orf142-I2) from the staphylococcal phage Twort, or a three nucleotide deletion mutant that deletes the intron's essential guanosine binding site, were introduced into the middle of the nes target sequence (highlighted), generating the conjugative plasmids pGO(I2) and pGO(dI2), respectively. (B) Transfer of the plasmids was tested using wild-type S. epidermidis RP62a as recipient. High conjugation efficiencies were obtained for pGO(I2) but not for pGO(dI2).

FIG. 8 shows mixed transformations. (A) The plasmids pLM314i, pLM314d, pLM317i, and pLM317d containing wild-type and mutant nes target sites were used to transform S. epidermidis in pair-wise combinations to allow for internally controlled transformation experiments. PCR primers used to amplify the target region in the transformants are shown at the bottom. The mutations in the pLM317 plasmids (highlighted in grey) include one that introduces a diagnostic SphI restriction site (underlined) that is absent from the pLM314 plasmids. (B) S. epidermidis RP62a and the isogenic Δcrispr mutant LAM104 were transformed with a mix containing equal amounts of pLM314d and pLM317d or pLM314i and pLM317i DNA, as indicated at the bottom. Transformation efficiency was calculated as cfu/μg DNA. (C) Primers P86 and P87 were used to amplify a 553 by PCR product from 10 colonies (lanes 1-10) obtained in each of the transformations shown in (B). DNA was cut with SphI and separated by 1.5% agarose gel electrophoresis. Purified pLM314 (lane “314”) and pLM317 (lane “317”) plasmid DNAs were used as controls. pLM317d (gels A and B) PCR product digestion generates two fragments of 222 and 331 bp. pLM317i (gels C and D) PCR product digestion generates two fragments of 328 and 225 bp.

FIG. 9 shows CRISPR loci from S. epidermidis RP62a and E. coli K12; colored boxes: CRISPR spacers; white boxes: CRISPR repeats; black boxes: CRISPR leader sequences. Flanking cas/csm/cse protein-coding genes that function in the CRISPR pathway are also depicted.

FIG. 10 shows an exemplary schematic of CRISPR interference. The CRISPR locus is depicted with repeats given as diamonds and spacers as boxes. The repeats and spacers are transcribed as pre-crRNA precursor that is processed into monomeric crRNA units, each containing a single spacer sequence. When the cell encounters foreign DNA with a sequence that matches a spacer, interference ensues via DNA targeting.

FIG. 11 shows protection of nes target by spacer flanking sequences: a) direct repeats (DR1-4, purple boxes) and spacers (1-3, colored boxes) of the S. epidermidis RP62a CRISPR locus were cloned into pC194 generating pCRISPR(wt) and its deletion variant, pCRISPR(del); b) pNes(wt) and pNes(mut) contain wild-type and mutated nes target sequence of pG0400 (highlighted in yellow, with mutations in red) and constitute positive and negative controls for CRISPR interference, respectively. 5′ and 3′ flanks of the wild-type nes target were replaced by upstream and downstream sequences of spc1 (highlighted in purple) in pNes(5′DR,8) and pNes(3′DR,15) constructs, respectively; c) each of the nucleotides upstream of nes target was replaced by the corresponding one from the region upstream of spc1 (highlighted in purple). The G at position −2 was also changed to C and T (in red). All plasmids were transformed into S. epidermidis RP62a and its isogenic Δcrispr mutant, LAM104. The average of at least three independent measures of the transformation efficiency (determined as cfu/μg DNA) is reported and error bars indicate 1 s.d.

FIG. 12 shows complementarity between crRNA and target DNA flanking sequences is required for protection; a) schematic of the complementarity between the flanking sequences of crRNA (top, highlighted in purple) and target DNA (bottom). The red box indicates the nucleotides mutated in the experiments shown in b; b and c) conjugation assays of pG0400 and its mutant variants, using as a recipient the Δcrispr strain LAM104 harboring different pCRISPR plasmids. Mutations are shown in red. Conjugation efficiency was determined as transconjugant cfu/recipient cfu; the average of at least 3 independent experiments is reported.

FIG. 13 shows mutations in upstream flanking sequences of CRISPR spacers elicit autoimmunity; a) deletions were performed in the flanking repeats of spc1: the 3′ half of DR2, the 5′ half of DR2, and all of DR1 were deleted from pCRISPR(wt) in pDR2(3′del), pDR2(5′del) and pDR1(del), respectively. b and c) substitutions (red) were introduced in the 5′ flanking sequence (highlighted in purple) of spc1 (highlighted in yellow), generating different pDR1 variants that were tested by transformation. All plasmids were transformed into S. epidermidis RP62a and LAM104 strains. The average of at least three independent measures of the transformation efficiency (determined as cfu/μg DNA) is reported and error bars indicate 1 s.d.

FIG. 14 shows requirements for targeting and protection during CRISPR immunity; a) in S. epidermidis, targeting of a bona-fide target is enabled by a lack of base pairing between the upstream flanking sequences of the crRNA spacer and target DNA. Formation of at least 3 base pairs at positions −4, −3 and −2 eliminates targeting. b) Full complementarity between the S. epidermidis CRISPR locus and the crRNA 5′ terminus results in protection. Disruption of at least two consecutive base pairs at positions −4,−3 or −3,−2 is required for loss of protection. c, General model for the prevention of autoimmunity in CRISPR systems. The ability of crRNA termini (5′, 3′, or both) to base pair with potential targets provides a basis for the discrimination between self and non-self DNA during CRISPR immunity.

FIG. 15 shows that repeat sequences present upstream of spc1 protect the nes target from CRISPR interference. a, Conservation of nes target and CRISPR spc1 flanking sequences. While target and spacer sequences (highlighted) are identical, three bases are conserved at positions −5, −4 and −3 and +1, +6 and +9 between the nes target and spc1 upstream and downstream flanking sequences, respectively (indicated by asterisks). b, Sequences flanking the 5′ or 3′ end of the nes target (highlighted) were replaced by the corresponding direct repeat (DR) sequences (highlighted) that flank spc1 in the CRISPR locus. 15 nt upstream or downstream of spc1 were introduced at the 5′ or 3′ end of the nes target in pNes(5′DR,15) or pNes(DR3′,15), respectively. Subsets of the sequences introduced into pNes(5′DR,15) were re-introduced upstream of the nes target, the nearest 8 nt to spc (nt 8-1) and the distal 7 nt (nt 15-9) generated pNes(5′DR,8-1) and pNes(5′DR,15-9), respectively. c, Individual mutations were also introduced in the downstream region of the nes target. Each nucleotide at positions +1 to +9 following the 3′ end of the nes target was substituted for the complementary base. Similar to pNes(DR3′,15), none of these mutation altered CRISPR interference of the respective plasmid. In all cases, sequences containing the nes target and the described changes were cloned into pC194 and the corresponding plasmids were transformed into S. epidermidis RP62a and its crispr isogenic mutant, LAM104. pNes(wt) and pNes(mut) were used as positive and negative controls for CRISPR interference, respectively. The average of at least three independent measures of the transformation efficiency (determined as cfu/ug DNA) is reported and error bars indicate 1 s.d.

FIG. 16 shows the effect of pG0400 mutations at position −2 during CRISPR conjugation interference. a, Mutations introduced in pG0400. Guanosine at position −2 (G-2, two nucleotides upstream of the start of the nes target sequence in pG0400) was mutated to adenosine (G-2A, introducing the nucleotide present at the corresponding position upstream of spc1, highlighted in purple), cytosine and thymidine (G-2C and G-2T, respectively; red font and highlighted in purple). These mutations change a glutamate residue of the Nes protein (E635) to lysine (K), glutamine (Q) and to a stop codon (*), respectively. pG0400(mut) contains mutations (red) in the nes target that do not alter the encoded protein2. b, Conjugative plasmids were tested using S. epidermidis RP62a and LAM104 as recipients. Conjugation efficiency was determined as transconjugant cfu/recipient cfu; the average of at least 3 independent experiments is reported and error bars indicate 1 s.d. Plasmids pG0400(wt) and pG0400(mut) were used as positive and negative controls of CRISPR interference2. pG0(G-2T) was unable to transfer into the S. epidermidis crispr strain LAM104, indicating that truncation of the Nes protein prevented proper nickase function during conjugation4; this plasmid was not further analysed.

FIG. 17 shows complementarity between crRNA and target DNA flanking sequences is required for protection. Conjugational transfer of pG0400 and its mutant variants into S. epidermidis crispr strain LAM104. Recipient cells contained different pCRISPR complementing plasmids. Red font indicates mutations introduced in the upstream flanking sequence (highlighted in purple) of spc1 (highlighted in yellow). Panels a-h show detailed results for each of the pCRISPR plasmids analysed in FIG. 10. Conjugation efficiency was determined as transconjugant cfu/recipient cfu; the average of at least 3 independent experiments is reported. Note that all plasmids were able to restore CRISPR interference in LAM104 cells with at least one of the conjugative plasmids, indicating that they all express functional crRNAs.

### DETAILED DESCRIPTION OF EMBODIMENTS

The present invention provides methods, systems, and compositions for interfering with the function and/or presence of a target DNA sequence in a eukaryotic cell (e.g., located in vitro or in a subject) using crRNA and CRISPR-associated (cas) proteins or cas encoding nucleic acids. The present invention also relates to a method for interfering with horizontal gene transfer based on the use of clustered, regularly interspaced short palindromic repeat (CRISPR) sequences.

CRISPR sequences are present in ˜40% of all eubacterial genomes and nearly all archaeal genomes sequenced to date, and are composed of short (˜24-48 nucleotide) direct repeats separated by similarly sized, unique spacers (Grissa et al. BMC Bioinformatics 8, 172 (2007), herein incorporated by reference in its entirety). They are generally flanked by a set of CRISPR-associated (cas) protein-coding genes that are important for CRISPR maintenance and function (Barrangou et al., Science 315, 1709 (2007), Brouns et al., Science 321, 960 (2008), Haft et al. PLoS Comput Biol 1, e60 (2005), herein incorporated by reference in their entirety). In Streptococcus thermophilus (Barrangou et al., Science 315, 1709 (2007), herein incorporated by reference in its entirety) and Escherichia coli (Brouns et al., Science 321, 960 (2008), herein incorporated by reference in its entirety). CRISPR/cas loci have been demonstrated to confer immunity against bacteriophage infection by an interference mechanism that relies on the strict identity between CRISPR spacers and phage target sequences, although the present invention is not limited to any particular mechanism of action and an understanding of the mechanism of action is not necessary to practice the present invention. CRISPR spacers and repeats are transcribed and processed into small CRISPR RNAs (crRNAs) (Tang et al., Proc. Natl. Acad. Sci. USA 99, 7536 (2002), Tang et al., Mol Microbiol 55, 469 (2005), herein incorporated by reference in their entireties).

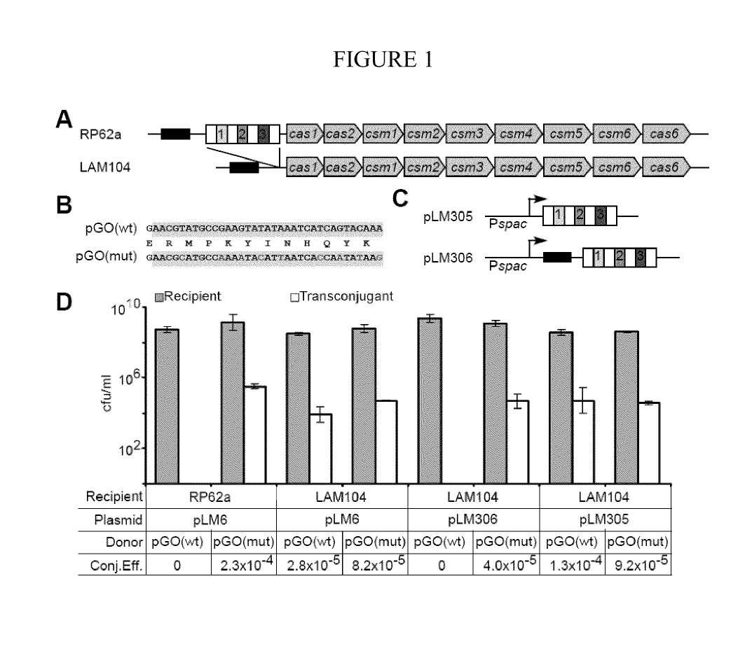

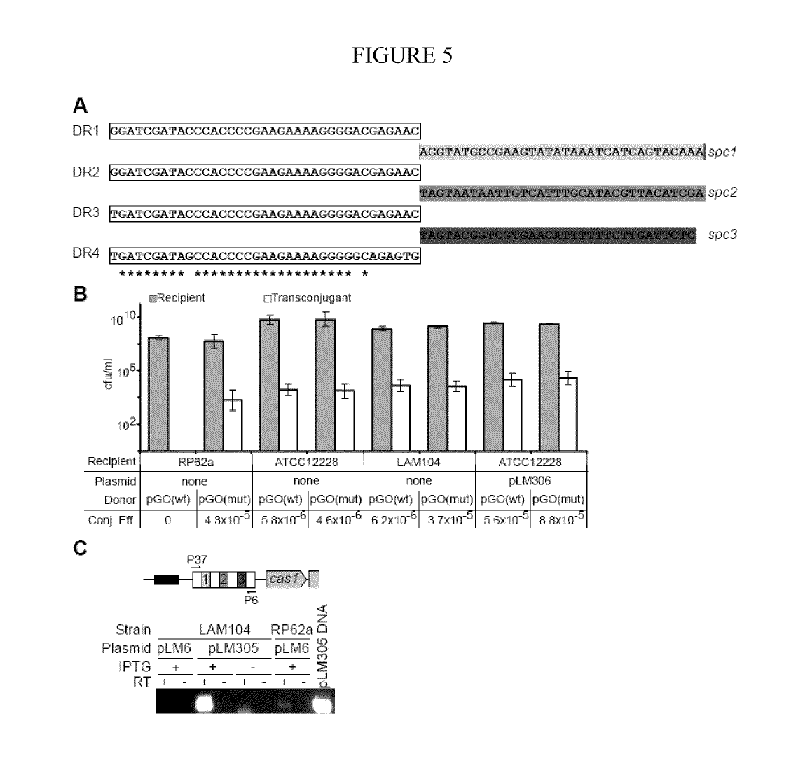

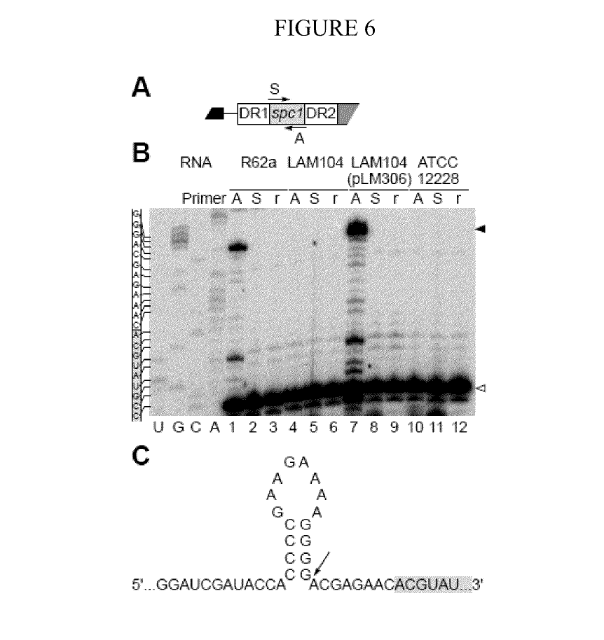

Along with S. aureus, S. epidermidis strains are the most common causes of nosocomial infections (Lim, S. A. Webb, Anaesthesia 60, 887 (2005), Lowy. N. Engl. J. Med. 339, 520 (1998), von Eiff, et al. Lancet Infect. Dis. 2, 677 (2002), herein incorporated by reference in their entireties), and conjugative plasmids can spread from one species to the other. While the S. epidermidis strain ATCC12228 (Zhang et al., Mol. Microbiol. 49, 1577 (2003), herein incorporated by reference in its entirety) lacks CRISPR sequences, the clinically isolated strain RP62a (Gill et al., J. Bacteriol. 187, 2426 (2005), herein incorporated by reference in its entirety) contains a CRISPR locus composed of three spacers and four repeats (SEE FIGS. 1A and 5A). One spacer sequence (spc3) has no matches in Genbank, another (spc2) matches a sequence present in the staphylococcal phage PH15 (Daniel. J Bacteriol 189, 2086 (2007), herein incorporated by reference in its entirety), and one (spc1) is homologous to a region of the nickase (nes) gene found in all staphylococcal conjugative plasmids sequenced so far, including those present in MRSA and VRSA strains (Climo et al. J. Bacteriol. 178, 4975 (1996), Diep et al., Lancet 367, 731 (2006), herein incorporated by reference in their entireties). The CRISPR repeat-spacer region is preceded by a ˜300 base pair (bp), A/T-rich, but otherwise non-conserved “leader” sequence that is observed at all CRISPR loci (Jansen. Mol. Microbiol. 43, 1565 (2002), Lillestøl et al. Archaea 2, 59 (2006), herein incorporated by reference in their entireties). A set of nine cas genes lies immediately downstream of the repeats and spacers (SEE FIG. 1A). The expression of spc1 crRNA in S. epidermidis RP62a but not ATCC12228 was confirmed by primer extension analysis of total RNA (SEE FIG. 6). As has been reported for other CRISPR systems, the 5′ end of the spc1 extension product maps to an apparent processing site at the base of a potential stem-loop structure in the preceding repeat (SEE FIG. 6C). Evidence of similar processing was obtained for spc2 and spc3 crRNAs.

Experiments conducted during development of embodiments of the present invention demonstrate that CRISPR interference acts at the DNA level, and therefore differs fundamentally from the RNAi phenomenon observed in eukaryotes and to which CRISPR activity was originally compared (Makarova et al. Biol. Direct. 1, 7 (2006), herein incorporated by reference in its entirety). Cas3, the primary candidate CRISPR effector protein in E. coli, contains domains predicted to confer nuclease and helicase functions, representing the minimal activities necessary for an RNA-directed dsDNA degradation pathway. An exemplary DNA targeting mechanism for CRISPR interference implies the presence of a system to prevent the targeting of the encoding CRISPR locus itself. The ability to direct the destruction of any given 24-48 nucleotide DNA sequence in a highly specific and addressable manner has considerable functional utility, particularly functioning outside of its native bacterial or archaeal context.

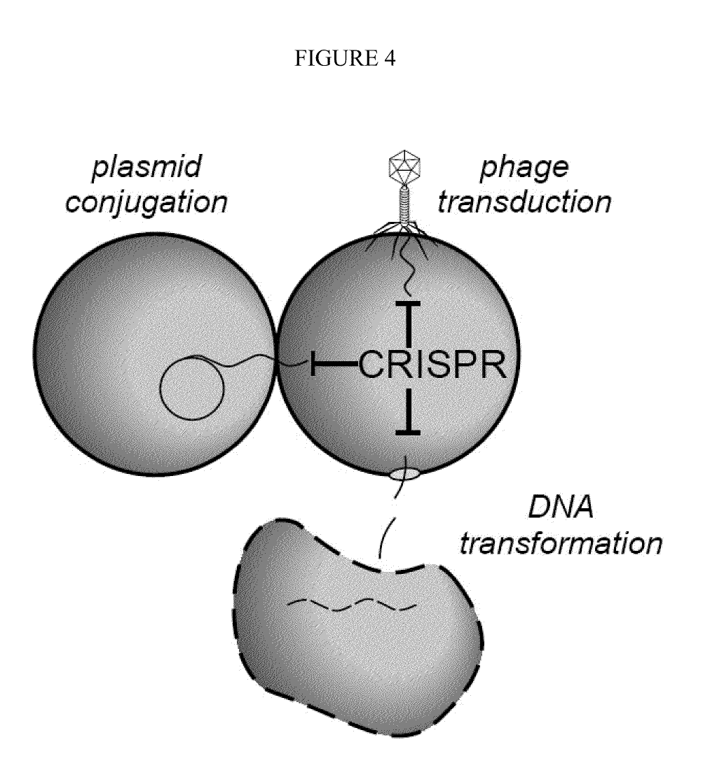

Experiments conducted during development of the present invention have shown that CRISPR loci provide immunity against bacteriophage infection and therefore should also prevent the exchange of bacterial DNA by transduction. By demonstrating that CRISPR interference abrogates plasmid conjugation and transformation, it has been demonstrated that CRISPR systems have a general role in the prevention of HGT (SEE FIG. 4) and therefore help bacterial strains maintain their respective genetic identities. In addition, in the particular case of S. epidermidis RP62a, the anti-conjugation CRISPR interference may prevent the acquisition of conjugative plasmids that, in the absence of selection, would impose an unnecessary energetic burden on the host cell. CRISPR function provides a barrier to gene transfer between members of the same genera or species that share restriction-modification enzymes, among which mobile DNA elements would otherwise move freely. It is contemplated that this may be the reason for the presence of CRISPR systems in some bacterial species and strains, although the present invention is not limited to any particular mechanism of action and an understanding of the mechanism of action is not necessary to practice the present invention. A primary difference between restriction-modification systems and CRISPR interference is that the latter can be programmed by providing a suitable effector crRNA. CrRNAs and CRISPR interference, manipulated in a clinical setting, provides a means to impede the ever-worsening spread of antibiotic resistance genes in staphylococci and other bacterial pathogens.

Thus, in some embodiments, the present invention provides compositions and methods for providing interference of horizontal gene transfer based on clustered, regularly interspaced short palindromic repeat (CRISPR) sequences. In some embodiments, CRISPR loci confer sequence-based, RNA-directed resistance against gene transfer (e.g. horizontal gene transfer from, e.g. viruses and plasmids). In some embodiments, CRISPR interference functions by targeting DNA molecules, although the present invention is not limited to any particular mechanism of action. In some embodiments, CRISPR machinery target DNA molecules within cells (e.g. eukaryotic, eubacteria, archaebacteria) in the presence of appropriate CRISPR RNAs. In some embodiments, the present invention provides and an addressable and readily reprogrammable DNA-targeting capability in eukaryotes such as yeast, metazoans, humans, non-human primates, mammals, canines, rodents, felines, bovines, equines, porcines, etc. In some embodiments, the present invention provides compositions and methods employing organisms engineered with heterologous nucleic acid sequences that alter the organism's susceptibility to receiving horizontal transfer of genetic material. In some embodiments, CRISPR loci of the present invention provide sequence-based, RNA-directed resistance against horizontal gene transfer from bacteria, viruses, and plasmids. CRISPR loci confer sequence-based, RNA-directed resistance against gene transfer (e.g. horizontal gene transfer from, e.g. viruses, bacteria, and plasmids) in bacteria, eubacteria, and eukaryotes. In some embodiments, the present invention finds utility in biotechnology and medicine.

In some embodiments, the present invention provides compositions and methods employing organisms engineered with heterologous nucleic acid sequences (e.g., nucleic acid sequences that are not native to the organisms either in terms of their presence in the organism or their location in the organism) that alter the organism's susceptibility to receiving horizontal transfer of genetic material. Such engineered organisms find use in commercial/industrial settings (e.g., industrial microbiology applications), research settings (e.g., basic research, drug screening, and the like), or therapeutic and medical settings.

In some embodiments, the present invention provides controlled, targeted genome manipulation in plants, animals, and their respective pathogens (e.g. for us in medicine, biotechnology, agriculture, etc.). Previous method for the alteration of specific genes (e.g. within multi-gigabase genomes) are limited. In some embodiments, the present invention provides efficient targeted modification (e.g. gene disruption, gene correction, insertion, etc.). In some embodiments, the present invention provides sufficient frequency to allow the isolation of a desired genotype without direct phenotypic selection. In some embodiments, the present invention provides specific targeted modification (e.g. modification of a single predetermined site within a genome). In some embodiments, the present invention provides targeted modification of a specific allele of a heterozygous locus. In some embodiments, gene modification is targeted by Watson-Crick pairing. In some embodiments, nucleic acid targeting provided targeted modification through Watson-Crick pairing.

Experiments performed during development of embodiments of the present invention have demonstrated that CRISPR interference (Sorek et al. Nature Rev. Microbiol. 6, 181-186 (2008), herein incorporated by reference in its entirety), an adaptive defense system against foreign genetic elements in bacteria and archaea (Barrangou et al. Science 315, 1709-1712 (2007), herein incorporated by reference in its entirety), targets DNA directly (Marraffini & Sontheimer. Science 322, 1843-1845 (2008), herein incorporated by reference in its entirety). In some embodiments, CRISPR specificity is established by about 24- to 48-nucleotide (nt) sequences (e.g. 20-50 nt, 24-48 nt, 28-44 nt, 32-40 nt, 20-32 nt, 32-48 nt, etc.) within small CRISPR RNAs (crRNAs) that require a match with their target (Barrangou et al. Science 315, 1709-1712 (2007), herein incorporated by reference in its entirety). In some embodiments, CRISPR nucleotides require a perfect sequence match with a target nucleic acid (e.g. target DNA). Experiments conducted during embodiments of the present invention demonstrate that CRISPR interference increases phage resistance by >10⁷-fold in E. coli, indicating that CRISPR DNA targeting is very efficient and robust.

In some embodiments, the present invention provides the CRISPR machinery in eukaryotic cells to enable addressable genome targeting through the transient or sustained expression of a suitable crRNA. In some embodiments, CRISPR provides targeting by a complementary 24-48 RNA sequence, with no little or no tolerance for mismatches. In some embodiments, CRISPR provides targeting based on Watson-Crick complementarity. In some embodiments CRISPR machinery can be reprogrammed to target a different DNA sequence through the use of a different crRNA. In some embodiments, active CRISPR machinery within a cell (e.g. eaukaryotic cell) is used with one or more crRNA (e.g. 1, 2, 3, 4, 5 . . . 10 . . . 20 . . . 50 . . . 100 . . . 200 . . . 500 . . . 1000, etc.). In some embodiments, active CRISPR machinery within a cell (e.g. eaukaryotic cell) is used to target one or more target sequences (e.g. 1, 2, 3, 4, . . . 5 . . . 10 . . . 20 . . . 50 . . . 100 . . . 200 . . . 500 . . . 1000, etc.). In some embodiments CRISPR machinery targets multiple sites in a viral genome (e.g. to prevent mutational evasion from occurring). In some embodiments CRISPR machinery targets multiple genes within a single cell (e.g. bacterial cell, eukaryotic cell, etc.). In some embodiments, targeting multiple sites within a single gene increases knockout, template repair efficiency, or provides the removal of specific exons by targeting the flanking introns. In some embodiments, the present invention provides a robust, RNA-guided, addressable, and/or reprogrammable tool for genome manipulation outside of bacteria and archaea (e.g. in eukaryotes).

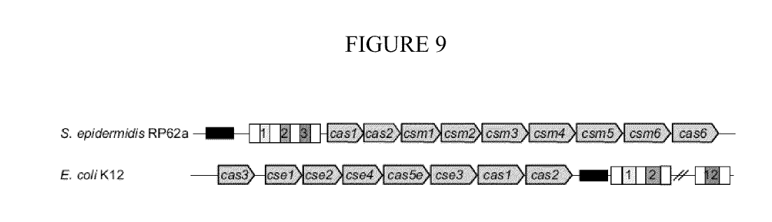

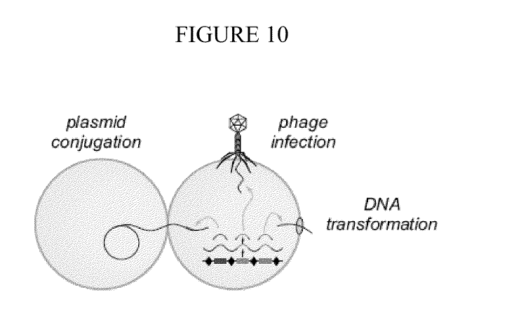

In some embodiments, the present invention utilizes a pathway found in many bacteria and archaea which confers resistance to bacteriophage infection and plasmid conjugation (Sorek et al. Nature Rev. Microbiol. 6, 181-186 (2008), Barrangou et al. Science 315, 1709-1712 (2007), Marraffini & Sontheimer. Science 322, 1843-1845 (2008), Brouns et al. Science 321, 960-964 (2008), herein incorporated by reference in their entireties). Resistance is specified by sequences that lie within clustered regularly interspaced short palindromic repeat (CRISPR) loci, which constitute a class of short (e.g. 24-48 nucleotide) direct repeats separated by unique spacer sequences of similar length (SEE FIG. 9). In some embodiments, the sequences of the unique spacers correspond to fragments of phages and plasmids that are endemic to the host species. In some embodiments, repeat/spacer units can be acquired from newly introduced foreign DNA, allowing the CRISPR locus to confer a form of adaptive immunity. In some embodiments, the repeats and spacers are transcribed and processed into small CRISPR RNAs (crRNAs) that specifically guide interference. In some embodiments, a single mismatch between the crRNA and its target greatly diminishes phage resistance (SEE FIG. 10). In some embodiments, protein-coding genes (e.g. CRISPR-associated (cas) genes) appear in conjunction with CRISPR repeats. In some embodiments, Cas genes are associated with CRISPR repeats. In some embodiments, Cas proteins are involved in the propagation and function of CRISPR loci (Sorek et al. Nature Rev. Microbiol. 6, 181-186 (2008), Barrangou et al. Science 315, 1709-1712 (2007), Brouns et al. Science 321, 960-964 (2008), Haft et al. PLoS Comput Bioi 1, e60 (2005), herein incorporated by reference in their entireties). In some embodiments, CRISPR interference is guided by an RNA component that identifies a target through Watson-Crick base pairing. In some embodiments, crRNAs target DNA directly. In some embodiments, proteins encoded by CRISPR/cas loci can be divided into core Cas proteins and subtype-specific proteins with more restricted phylogenetic distributions (e.g. the Csm and Cse proteins; SEE FIG. 9) (Haft et al. PLoS Comput Bioi 1, e60 (2005), herein incorporated by reference in its entirety). In some embodiments, considerable variation exists among the core Cas genes. In some embodiments, cas1 is universally present in CRSPR sequences. In some embodiments, Cas1 and Cas2 function in the acquisition and maintenance of new repeat/spacer units. In some embodiments, Cse1-5 proteins form a Cascade complex (CRISPR-associated Complex for antiviral defense) that functions in pre-crRNA processing. In some embodiments, Cse3 is the catalytic subunit and the only protein required for the generation of monomeric crRNAs (e.g. from multimeric precursor (e.g. via cleavage at a specific site within each repeat)) (SEE FIG. 10). In some embodiments, Cas3, is not required for crRNA accumulation but is essential for interference. In some embodiments Cas3 is an effector protein in E. coli. In some embodiments Cas3 comprises an HD nuclease domain and/or a DEAD-box helicase domain. In some embodiments, Cas3 utilizes an RNA guide (e.g. crRNA) to target double-stranded DNA. In some embodiments, the CRISPR pathway functions using species-specific Cas proteins. In some embodiments, the CRISPR pathway utilizes proteins which are homologous, analogous, non-homologous, non-related, functionally similar, and/or functionally dissimilar to the Cas proteins of E. coli (e.g. the CRISPR pathway in metazoans).

In some embodiments, the present invention provides RNA-directed DNA-targeting in eukaryotic cells (e.g. yeast, metazoan, human, etc.) through the exploitation of the CRISPR system. In some embodiments, the present invention applies the natural CRISPR pathways of bacteria and eubacteria as a technology to manipulate the complex genomes of plants and animals (e.g. humans), such as in knock-out experiments.

In some embodiments, the present invention finds utility in medicine, research, agriculture, veterinary medicine, and other fields. In some embodiments, the present invention comprises compositions, methods, kits, reagents, devices, and/or systems for use in the inhibition of horizontal gene transfer.

In some embodiments, the present invention provides compositions and methods for inhibiting horizontal gene transfer. In some embodiments, the present invention protects a cell, tissue, organ, or subject from the transfer of foreign genes (e.g. from plasmids, bacteria, viruses, etc.). In some embodiments, the present invention protects a bacteria (e.g. eubacteria or archaebacteria), eukaryote, metazoan, mammal, human, non-human primate, rodent, bovine, equine, porcine, feline, canine, etc. from horizontal gene transfer of foreign genes.

### EXPERIMENTAL

### Example 1

### Interference of Horizontal Gene Transfer

Along with S. aureus, S. epidermidis strains are the most common causes of nosocomial infections, and conjugative plasmids can spread from one species to the other. The S. epidermidis strain ATCC12228 lacks CRISPR sequences. The clinically isolated strain RP62a contains a CRISPR locus composed of three spacers and four repeats (SEE FIGS. 1A and 5A). One spacer sequence (spc3) has no matches in Genbank, another (spc2) matches a sequence present in the staphylococcal phage PH15, and one (spc1) is homologous to a region of the nickase (nes) gene found in all staphylococcal conjugative plasmids sequenced so far, including those present in MRSA and VRSA strains. The CRISPR repeat-spacer region is preceded by a ˜300 base pair (bp), A/T-rich, but otherwise non-conserved “leader” sequence that is observed at all CRISPR loci. A set of nine cas genes lies immediately downstream of the repeats and spacers (SEE FIG. 1A).

In experiments performed during developments of embodiments of the present invention, the expression of spc1 crRNA in S. epidermidis RP62a, but not ATCC12228, was confirmed by primer extension analysis of total RNA (SEE FIG. 6). The 5′ end of the spc1 extension product maps to an apparent processing site at the base of a potential stem-loop structure in the preceding repeat (SEE FIG. 6). Evidence of similar processing was obtained for spc2 and spc3 crRNAs.

Spc1 may prevent plasmid conjugation to or from S. epidermidis RP62a through an interference mechanism that relies on the sequence identity between the spacer and its target sequence in the plasmid. Experiments were performed to disrupt the sequence match by introducing nine silent mutations into the nes target in the conjugative plasmid pGO400, generating pGO(mut) (SEE FIG. 1B). PGO400 was selected because it provides a suitable marker (mupirocin resistance) for selection in S. epidermidis RP62a recipients. The mutations introduced during development of embodiments of the present invention into pGO(mut) alter the DNA and mRNA sequence but leave intact the amino acid sequence of the nickase protein, which is required in the donor cell for conjugative transfer. Both wild-type and mutant pGO400 were tested for transfer from S. aureus strain RN4220 (Kreiswirth et al., Nature 305, 709 (1983), herein incorporated by reference in its entirety) into either of the two S. epidermidis strains (SEE FIGS. 1D and 5B). While the conjugation frequency of both plasmids was similar for the CRISPR-negative ATCC12228 strain, only pGO(mut) was transferred into the CRISPR-positive RP62a strain, and with a frequency similar to that of wild-type pGO400 in the control ATCC12228 strain. In all conjugation assays, the genetic identity of the apparent transconjugants was confirmed by PCR. Experiments performed during developments of the present invention indicate that CRISPR interference can prevent plasmid conjugation in a manner that is specified by sequence identity between a spacer and a plasmid target sequence.

In experiments conducted during development of embodiments of the present invention, the four repeats and three spacers present in the S. epidermidis RP62a locus were deleted, generating the isogenic Δcrispr strain LAM104 (SEE FIG. 1A). Lack of crRNA expression in the mutant was confirmed by primer extension (SEE FIG. 6, lanes 4-6). The ability of LAM104 to act as recipient for the conjugative transfer of pGO400 was tested. Wild-type RP62a was refractory to pGO400 transfer, whereas the conjugation efficiency for the LAM104 strain was similar to that obtained for S. epidermidis ATCC12228 (SEE FIGS. 1D and 5B). As a control, pGO(mut) transfer was similar in both strains. To restore interference in the Δcrispr mutant, LAM104 was transformed with plasmid pLM305 or pLM306 (SEE FIG. 1C). Both of these plasmids contain the repeat-spacer region of the S. epidermidis RP62a CRISPR locus downstream of an IPTG-inducible promoter, but they differ in the amount of upstream flanking sequence included (61 vs. 700 by in pLM305 and pLM306, respectively). The appearance of the spc1 crRNA in the pLM306-containing cells was not IPTG-dependent, indicating that the upstream region in pLM306 includes a natural CRISPR promoter. In conjugation assays using wild type pGO400, pLM306 restored interference in strain LAM104 but pLM305 did not, even when expression of the repeats and spacers was induced by IPTG (SEE FIG. 5C). This suggests a role for the leader sequence or other upstream sequences in cis during CRISPR interference, although the present invention is not limited to any particular mechanism of action and an understanding of the mechanism of action is not necessary to practice the present invention. Introduction of pLM306 into the CRISPR-lacking strain ATCC12228 did not alter the conjugation efficiency of pGO400 (SEE FIG. 5C).

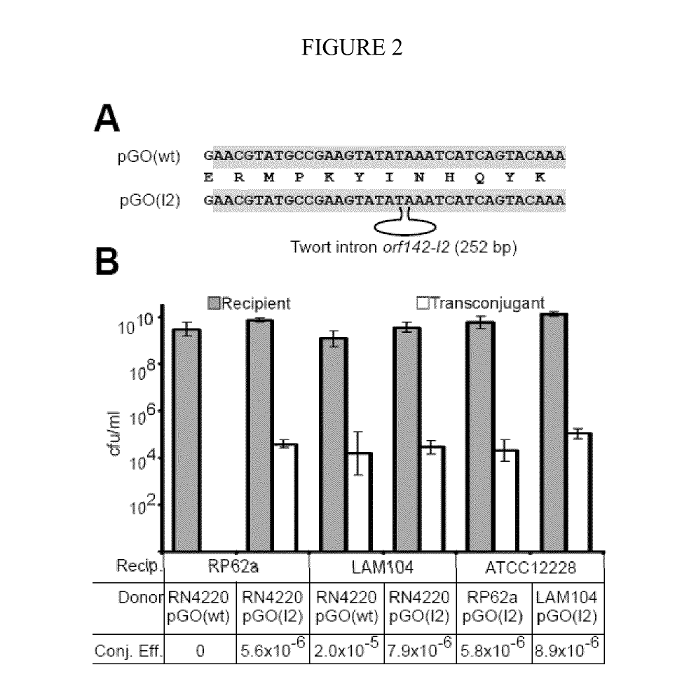

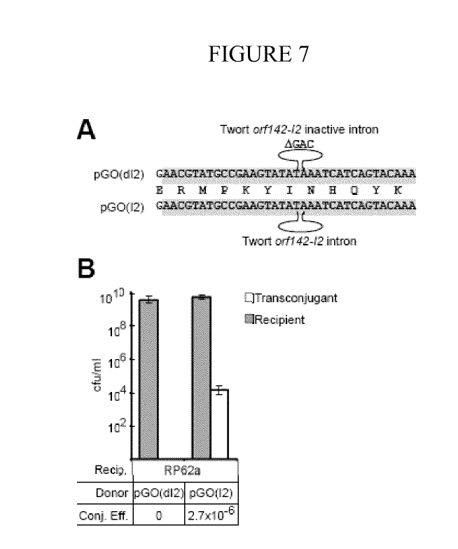

Experiments performed during developments of the present invention (SEE FIGS. 1 and 5) indicate that the CRISPR locus limits the ability of S. epidermidis RP62a to act as a recipient of conjugative plasmids, and therefore suggest that spc1-directed interference does not target the nes transcript. The orientation of spc1 leads to the expression of a crRNA that is identical with, rather than complementary to, the nes mRNA target sequence, providing no evidence for the expression of RNA from the opposite strand (SEE FIG. 6B). An alternative to mRNA targeting is the possibility that the spc1 crRNA targets the incoming DNA. The target sequence of the pGO400 nes gene was interrupted with a self-splicing group I intron to disrupt the potential for DNA but not mRNA recognition. The orf142-I2 intron from the staphylococcal Twort phage was used, as it is a well-characterized intron that splices very efficiently and rapidly in staphylococci (Golden et al., Nat. Struct. Mol. Biol. 12, 82 (2005), Landthaler et al. Proc. Natl. Acad. Sci. USA 96, 7005 (1999), herein incorporated by reference in their entireties). The mutant conjugative plasmid, pGO(I2), lacks an intact spc1 target DNA sequence (SEE FIG. 2A), but the spc1 target sequence is regenerated in the nes mRNA after transcription and rapid splicing. Conjugation assays performed during development of the present invention revealed pGO(I2) was transferred to wild-type and Δcrispr strains with equal efficiencies (SEE FIG. 2B). This observation reflects an evasion of CRISPR interference when the intron is present in the plasmid, and therefore indicates that an intact target site is required in the nes DNA, but not mRNA, for interference to occur. To confirm that splicing is required for nes function, the conjugation of pGO(dI2), a derivative of pGO(I2) containing a three-nucleotide deletion within the intron that inactivates self-splicing (24) (SEE FIG. 7A). In contrast to pGO(I2), the mutant plasmid was unable to transfer to the RP62a strain (SEE FIG. 7B), indicating a lack of nickase activity in the presence of the unspliced intron.

The requirement for nes transcription, splicing, and translation in the donor cell during conjugation, and the ability to obtain RP62a transconjugants with the intron-containing pGO(I2) plasmid, allowed testing of the capacity of the CRISPR system to target intact, spliced nes mRNA by using RP62a as a pGO(I2) donor. The pGO(I2) conjugative transfer was just as efficient from RP62a as from the isogenic Δcrispr strain LAM104 (SEE FIG. 2), indicating that spliced, functional nes mRNA, which must be present for conjugation to occur, is not targeted during CRISPR interference. This provides strong evidence that DNA rather than mRNA is the likely crRNA target during CRISPR interference.

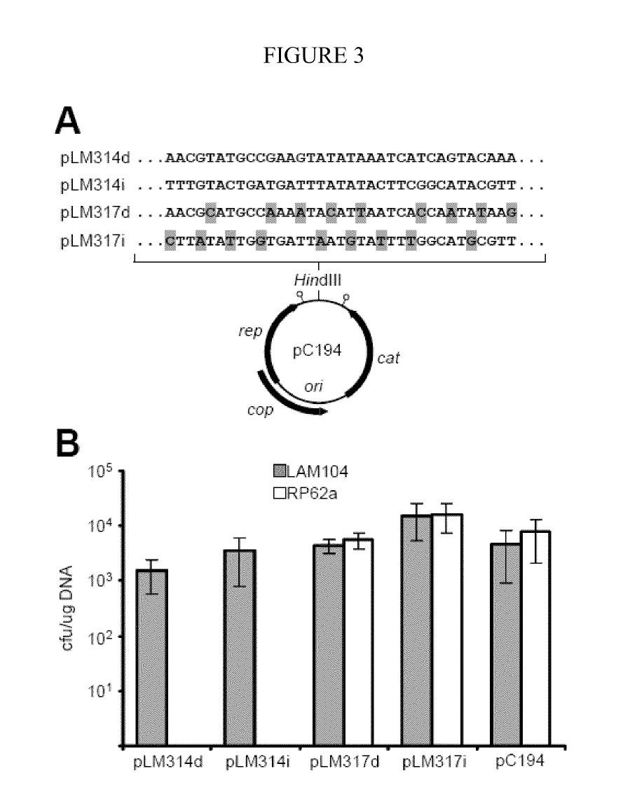

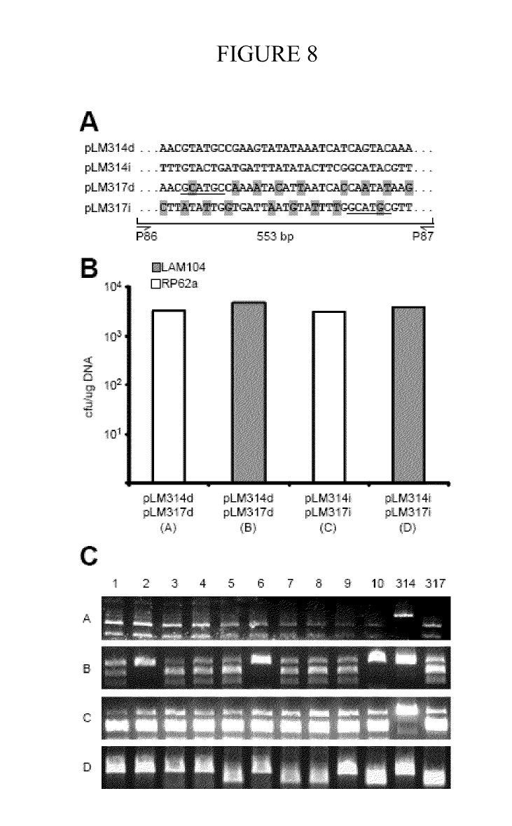

The CRISPR-mediated interference with phage and conjugative plasmid DNA molecules demonstrates that CRISPR systems function to prevent HGT, a function conceptually similar to that of restriction-modification systems (Tock and Dryden, Curr. Opin. Microbiol. 8, 466 (2005), herein incorporated by reference in its entirety). CRISPR loci should also prevent DNA transformation, the third mechanism of HGT besides bacteriophage transduction and plasmid conjugation. PGO(wt) and pGO(mut) nes target and flanking sequences (200 bp) were introduced in either orientation into the HindIII site of the staphylococcal plasmid pC194, generating pLM314 and pLM317, respectively (d, direct insertion; i, inverted insertion; SEE FIG. 3A). Plasmids were transformed by electroporation into wild-type RP62a and isogenic Δcrispr LAM104 strains. pC194, pLM317d and pLM317i were transformed into both strains (SEE FIG. 3B). pLM314d and pLM314i, however, were only transformed into the Δcrispr mutant, and with efficiencies similar to those of the pLM317 and pC194 plasmids. In addition, pLM314/pLM317 mixed transformations of RP62a or LAM104 strains (SEE FIG. 8) were performed to test transformation in an internally controlled fashion. Ten of the transformants obtained were analyzed by PCR for the presence of either pLM314 or pLM317. Regardless of the direction of the insert, only pLM317 was found among wild-type RP62a transformants, whereas both pLM314 and pLM317 were found in LAM104 transformant colonies, consistent with CRISPR interference specifically with the plasmid that carries the perfectly matched target sequence. Although plasmid electroporation differs from natural transformation, both processes rely on the incorporation of foreign, naked DNA into the bacterial cell (Dubnau, Annu. Rev. Microbiol. 53, 217 (1999), herein incorporated by reference in its entirety) and thus these results demonstrate that CRISPR systems can also block this primary route of HGT. The experiments performed during development of embodiments of the present invention provide evidence that crRNAs target DNA molecules. First, interference occurred regardless of the insert orientation in pLM314; this, combined with the lack of compelling evidence for CRISPR-derived double-stranded RNA (SEE FIG. 6B), is consistent with spc1 targeting either DNA strand rather than a unidirectional transcript. Second, the target sites in the pLM314 and pLM317 plasmids are located between the transcriptional terminators of the rep and cat genes (SEE FIG. 3A) (27, 29, 30), minimizing the likelihood that the target sequence is transcribed. The HindIII site region of pC194 has been interrupted or deleted in numerous reports without affecting the replication and maintenance of the plasmid (Gros et al. EMBO J. 6, 3863 (1987), Horinouchi and Weisblum. J. Bacteriol. 150, 815 (1982), herein incorporated by reference in their entireties.

### Example 2

### Interference in Eukaryotes

During development of embodiments of the present invention, eubacterial species (e.g. E. coli, S. epidermidis, and Streptococcus thermophilus) were used as model systems for CRISPR function for mechanistic analysis. Commercial gene synthesis can be used to obtain yeast-codon-optimized ORF cassettes for the insertion of cas3 and cse1-5 into a range of expression vectors (e.g. inducible and constitutive, plasmid-based and integrated, etc.). The N- and C-terminally tagged version of each Cas/Cse protein are known to be functional. In some embodiments, epitope tags and nuclear localization signals (NLSs) are used on proteins for analyses and to provide subcellular localization. Expression and localization is determined by western blot and immunofluorescence under a range of conditions to identify those that are most effective. The Cascade subunits are analyzed to determine whether they co-immunoprecipitate (co-IP) when expressed in various combinations. The proteins are coexpressed with multimeric repeat-spacer transcripts, with natural phage spacers, known to be valid Cascade substrates. Northern blots, primer extensions, and inverse RT-PCR assays are used to determine whether the crRNAs are expressed and stably processed in a Cascade-dependent manner. Mature crRNAs are analyzed for co-IP with Cascade, as observed in E. coli.

To streamline CRISPR function in yeast, crRNA constructs are produced in which the normal termini of mature crRNAs can be generated independent of Cascade. In some embodiments, cis-acting ribozymes (Ferre-D'Amare & Doudna. Nucleic Acids Res 24, 977-978 (1996), herein incorporated by reference in its entirety), most of which leave 5′-hydroxyl and 2′,3′-cyclic phosphate ends are used to generate crRNAs for use in yeast. In some embodiments, crRNAs are produces with 5′-monophosphate and 3′-hydroxyl ends to provide adequate substrates for T4 RNA ligase (e.g. as endogenous crRNAs are) (Brouns, et al. Science 321, 960-964 (2008), Hale et al. RNA 14, 2572-2579 (2008), herein incorporated by reference in their entireties). In some embodiments, crRNAs are generated by appending known substrates for native yeast RNA-cleaving enzymes (e.g. RNase P and Rnt1 p (tRNAs and specific stem-loops, respectively)) that leave desired termini. The substrates for these and other enzymes are well defined and therefore readily exploitable.

Nuclear Cas3 is expressed an tested for crRNAs co-immunoprecipitation (e.g. when the crRNAs are made in a Cascade-dependent or -independent manner). In some embodiments, productive Cas3/crRNA loading may be coupled to Cascade processing. Targeting tests that could be performed employ a well-established assay for the frequency of DSB induction that employs a strain carrying a URA3 cassette integrated adjacent to a ura3 allele (Sugawara & Haber. Mol Cell Bioi 12, 563-575 (1992), herein incorporated by reference in its entirety). The presence of URA3 renders the cell sensitive to 5-fluoroorotic acid (5-FOA). Homologous recombination between the two alleles causes a loss of URA3, leading to 5-FOA resistance. This type of recombination occurs at a very low spontaneous rate (˜10⁻⁵-10⁻⁶), and this rate increases by several orders of magnitude upon the introduction of a DSB between the alleles. One or more crRNAs that target the genomic sequences are introduced between the two alleles, and the frequency of 5-FOA resistance is measured. This frequency rises in a Cas3 and cognate crRNA-dependent manner when the CRISPR system is functional. Due low background, the assay is a more sensitive measurement of DSB induction than related assays that score plasmid loss in response to DSB induction. The low background and quantitative nature of the assay are important for detecting modest but reproducible frequencies of crRNA targeting. Target sites are chosen based on minimal similarity to any other sequence in the yeast genome, along with proximity to the short (4-5-nt) flanking CRISPR motif for crRNA targeting (Deveau et al. J Bacteriol 190, 1390-1400 (2008), Horvath et al. J Bacteriol 190, 1401-1412 (2008), herein incorporated by reference in their entireties). This sequence has been defined in S. thermophilus and S. epidermidis. A requirement for CRISPR motif proximity does not significantly limit targeting options given the frequency of such short sequences.

Growth rates can be measured in a range of conditions to detect toxicity associated with the expression of Cas proteins with or without exogenous crRNAs. Toxicity, if it exists, is due to off-target effects during true crRNA targeting or the spurious recruitment of “cryptic crRNAs” derived from yeast transcripts. The fidelity of targeting is assessed by measuring DSB frequency with crRNAs that have one or a few mismatches in the spacer sequence. This data should indicate that the fidelity of CRISPR interference in bacteria is recapitulated in eukaryotes. If mismatches are tolerated to any degree, fidelity is characterized in detail to define the capabilities of the system and its capacity for further exploitation.

CRISPR RNA-directed DNA targeting is tested for efficacy in animals by exploiting the genetic and phenotypic tools that are available for the Drosophila melanogaster. The fly genome is more than an order of magnitude larger than that of yeast. ORF constructs that are codon-optimized for Drosophila expression are constructed for the minimal complement of Cas proteins found to be essential for crRNA-guided DNA targeting in yeast. Epitope tags and NLS sequences are appended to the Cas constructs. The constructs are cloned behind the Drosophila heat-shock promoter and the Pacman transgenesis system to introduce the genes into defined, expression-validated sites in the fly genome (Venken et al. Science 314, 1747-1751 (2006), herein incorporated by reference in its entirety). Transcription of the transgenes is induced by heat shock, and protein expression and localization is assayed by western blots and immunofluorescence. Known interactions among the Cas proteins are assayed by co-IP assays. Developmental defects in comparison with nonheat-shocked siblings or heat-shocked controls that lack the transgenes are assayed for. A range of heat shock regimens are tested with varied temperature, duration, or both to identify those conditions that provide the best balance between expression and toxicity (if any toxicity is detected; otherwise only expression is optimized). A separate Pacman heat-shock-inducible transgene construct that drives the expression of crRNAs with phage spacer sequences that have no significant matches in the fly genome is constructed. This transgene is inserted into a separate site, and heat-shock-induced pre-crRNA transcription is confirmed. The pre-crRNA transgene is crossed into the background of the Cas-protein-expressing transgenes, and the pre-crRNA is processed into crRNAs that associate with Cas proteins.

DNA targeting is tested by introducing into the Cas-expressing background a new heatshock-inducible pre-crRNA construct with spacers corresponding to the rosy (ry) gene. The Cas- and cognate crRNA-dependent frequency of induction of the ry eye-color phenotype in the progeny of heat-shocked males or females crossed to flies carrying known ry mutations is assessed. New apparent ry mutants recovered with this non-complementation approach, are examined at the molecular level to confirm mutagenesis and characterize the nature of the crRNA-induced allele. The consistency of the approach is tested to ensure that mutant alleles can be obtained based solely on molecular screening (e.g. PCR and “surveyor” nuclease CEL-I cleavage (Till et al. Nucleic Acids Res 32, 2632-2641 (2004), herein incorporated by reference in its entirety)) rather than mutant phenotype. The fidelity of targeting in flies is tested by characterizing the effects of crRNA mutants.

The CRISPR pathway system is used for the generation of targeted mutants in mammalian cells (e.g. human cells). Nuclear Cas protein is generated and validated in the mammalian cells, and crRNA expression is validated in the mammalian cells. Expression is achieved by transfection, since transient targeting function would suffice to leave a permanent genomic mark. Interactions of the Cas proteins with each other and with crRNAs is monitored, as is pre-crRNA processing (e.g. if Cascade cannot be bypassed by cellular processing activities). Cell viability is examined to detect possible toxicity. Functional tests involve the Cas3- and cognate crRNA-dependent targeting of the gene encoding dihydrofolate reductase (DHFR) (Santiago et al. Proc Natl Acad Sci USA 105, 5809-5814 (2008), herein incorporated by reference in its entirety), which is effectively diploid in these cells. Biallelic DHFR targeting is analyzed phenotypically based upon a requirement for hypoxanthine and thymidine in the culture medium. Pools of cells are examined by PCR/CEL-I assays to detect mutational events in the targeted regions. Mutants are characterized at the sequence level. Further analyses is performed to validate DHFR targeting.

### Example 3

### Self vs. Non-Self Discrimination

Bacterial strains and growth conditions. S. epidermidis RP62a (Gill et al. J. Bacteriol. 187, 2426-2438 (2005), herein incorporated by reference in its entirety) and LAM104 (Marraffini & Sontheimer. Science 322, 1843-1845 (2008), herein incorporated by reference in its entirety) and S. aureus RN4220 ((Kreiswirth et al. Nature 305, 709-712 (1983), herein incorporated by reference in its entirety)) strains were grown in brain-heart infusion (BHI) and tryptic soy broth media, respectively. When required, the medium was supplemented with antibiotics as follows: neomycin (15 μg/ml) for selection of S. epidermidis; chloramphenicol (10 μg/ml) for selection of pC194-based plasmids; and mupirocin (5 μg/ml) for selection of pG0400-based plasmids. E. coli DH5α cells were grown in LB medium, supplemented with ampicillin (100 μg/ml) or kanamycin (50 μg/ml) when necessary.

DNA cloning. Plasmids used during development of embodiments of the present invention were constructed by cloning CRISPR or nes sequences into the HindIII site of pC194 (Horinouchi & Weisblum. J. Bacteriol. 150, 815-825 (1982), herein incorporated by reference in its entirety).

Conjugation and transformation. Conjugation and transformation were performed as described previously (Marraffini & Sontheimer. Science 322, 1843-1845 (2008), herein incorporated by reference in its entirety) with the following modification: transformations of S. epidermidis were recovered at 30° C. in 150 μl of BHI for 6 hs. Corroboration of the presence of the desired plasmid in transconjugants or transformants was achieved by extracting DNA of at least two colonies, performing PCR with suitable primers and sequencing the resulting PCR product.

CRISPR loci are present in ˜40% and ˜90% of sequenced eubacterial and archaeal genomes respectively (Grissa et al. BMC Bioinformatics 8, 172 (2007), herein incorporated by reference in its entirety), and confer adaptive immunity against bacteriophage infection and plasmid conjugation (Barrangou et al. Science 315, 1709-1712 (2007), Brouns et al. Science 321, 960-964 (2008), Marraffini & Sontheimer. Science 322, 1843-1845 (2008), herein incorporated by reference in their entireties). CRISPR loci evolve rapidly, acquiring new spacer sequences to adapt to highly dynamic viral populations (Andersson & Banfield. Science 320, 1047-1050 (2008), Deveau. et al. J. Bacteriol. 190, 1390-1400 (2008), van der Ploeg. Microbiology 155, 1966-1976 (2009), herein incorporated by reference in their entireties). These clusters are genetically linked to a conserved set of cas (CRISPR-associated) genes (Haft et al. PLoS Comput. Biol. 1, e60 (2005), Makarova et al. Biol. Direct. 1, 7 (2006), herein incorporated by reference in their entireties) that encode proteins involved in adaptation and interference. A CRISPR RNA (crRNA) precursor containing multiple repeats and spacers is processed into small crRNAs (Carte et al. Genes Dev. 22, 3489-3496 (2008), Hale et al. RNA 14, 2572-2579 (2008), Brouns et al. Science 321, 960-964 (2008), herein incorporated by reference in their entireties). Processing occurs within the repeats and results in crRNAs that contain a single spacer flanked at both ends by partial repeat sequences. CRISPR interference is directed by crRNAs and target specificity appears to be achieved by Watson-Crick pairing between the spacer sequence in the crRNA and the “protospacer” in the invasive DNA. However, this sequence match also exists between the crRNA and the CRISPR locus that encodes it.

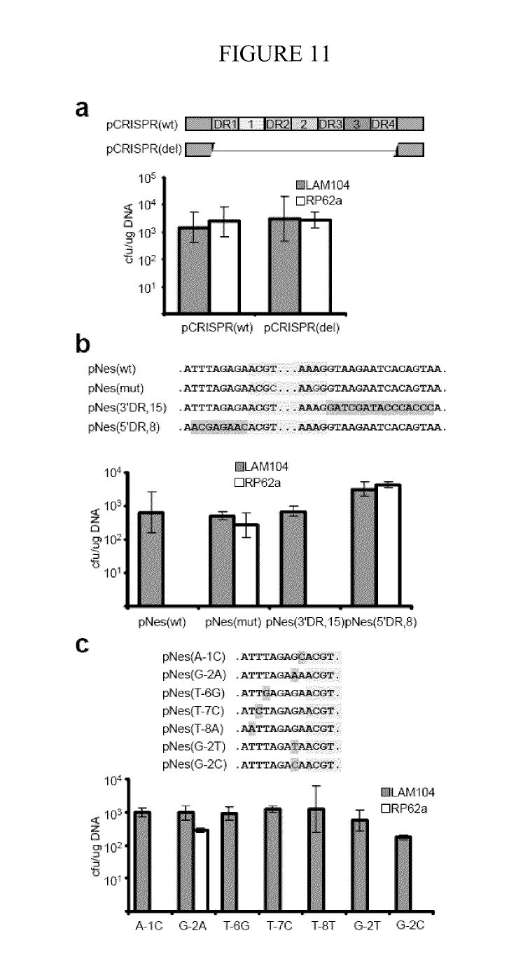

S. epidermidis RP62a (Gill et al. J. Bacteriol. 187, 2426-2438 (2005), herein incorporated by reference in its entirety) contains a CRISPR locus that includes a spacer (spc1) that is identical to a region of the nickase (nes) gene found in nearly all sequenced staphylococcal conjugative plasmids (SEE FIG. Y1a), including those that confer antibiotic resistance in methicillin- and vancomycin-resistant Staphylococcus aureus strains (Climo et al. J. Bacteriol. 178, 4975-4983 (1996), Diep et al. Lancet 367, 731-739 (2006), Weigel et al. Science 302, 1569-1571 (2003), Berg et al. J. Bacteriol. 180, 4350-4359 (1998), herein incorporated by reference in its entirety). It was previously demonstrated that the S. epidermidis CRISPR system limits conjugation between staphylococci and also prevents plasmid transformation. Introduction of nes protospacer-containing sequences from the conjugative plasmid pG0400 (Morton et al. Antimicrob. Agents. Chemother. 39, 1272-1280 (1995), herein incorporated by reference in its entirety) into the staphylococcal plasmid pC194 (Horinouchi &Weisblum. J. Bacteriol. 150, 815-825 (1982), herein incorporated by reference in its entirety) prevented transformation of that plasmid into RP62a, but not into an isogenic mutant (LAM104) lacking the repeat and spacer region of the CRISPR locus, demonstrating CRISPR-specific interference towards plasmid transformation. To test whether CRISPR spacers have an intrinsic ability to evade interference, the same approach was followed and the repeat/spacer sequences of the RP62a CRISPR locus was cloned, along with ˜200 base pairs (bp) from either side of the repeats and spacers, into pC194 (SEE FIG. 11a). The resulting plasmid, pCRISPR(wt), which contains three possible interference targets (spc1, spc2 and spc3), was transformed into wild-type and LAM104 Δcrispr cells. Unlike the nes protospacer-containing plasmid, pCRISPR(wt) transformation efficiency was similar in both strains and was also comparable to that of a plasmid lacking the repeats and spacers. These results indicate that the potential targets present in the CRISPR locus are specifically exempted from CRISPR interference.

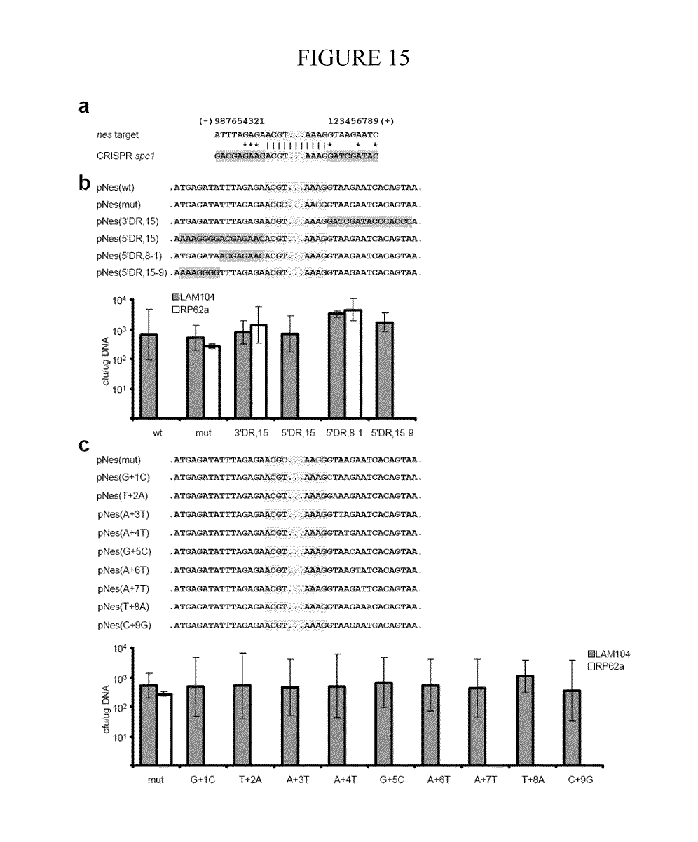

In some embodiments, the differences between flanking regions of spacers and targets (e.g the presence or absence of repeats) provides the basis for self/non-self discrimination. 15 by from either side of the nes target was replaced with the corresponding spc1-flanking repeat sequences. The resulting plasmids [pNes(5′DR,15) and pNes(3′DR,15)] were tested for CRISPR interference by transformation into wild-type and LAM104 Δcrispr cells (SEE FIGS. 11b and 15b). Only pNes(5′DR,15) escaped interference in wild-type cells, whereas both plasmids were transformed into LAM104, indicating that repeat sequences upstream of a target can protect that target from interference. Similar experiments performed during development of embodiments of the present invention further narrowed the protective region to the eight by closest to the target (SEE FIGS. 11b and 15b). The interference-insensitive plasmid pNes(5′DR,8-1) contains five mutations in the nes upstream sequence, since three of the eight by [the 5′-AGA-3′ sequence from position −5 (i.e., 5 bps upstream of the start of the nes protospacer) to −3) are shared with spc1 5′ flank (SEE FIG. 15a). Each of these five mutations was introduced individually and their effects on CRISPR interference were measured by plasmid transformation (SEE FIG. 11c). Only the guanosine-to-adenosine change at position −2 (G-2A) conferred protection. These results demonstrate that repeat sequences upstream of spacers prevent CRISPR interference, and point to position −2 as an important determinant of this effect.

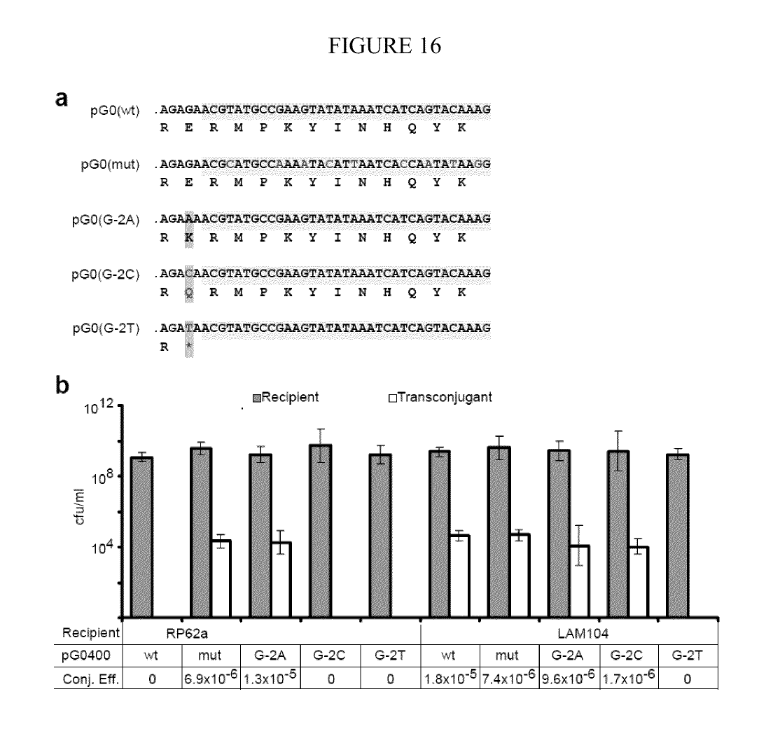

Short (2-4 bp), conserved sequence elements called “CRISPR motifs” or “protospacer adjacent motifs” (PAMs) have been found to exist in the vicinity of protospacers in other CRISPR systems, and mutations in these motifs can compromise interference (Deveau et al. J. Bacteriol. 190, 1390-1400 (2008), Semenova et al. FEMS Microbiol. Lett. (2009), Mojica et al. Microbiology 155, 733-740 (2009), herein incorporated by reference in their entireties). Transformation efficiency of plasmids carrying the mutations G-2C and G-2T upstream of the nes target was tested. Surprisingly, unlike the G-2A mutation, C and T transversions at this position had no effect on transformation efficiency (SEE FIG. 11c). This result was corroborated in a conjugation assay using pG0400 mutants containing G-2A or G-2C changes (SEE FIG. 16) and excludes the possibility that a G at position −2 is simply a crucial CRISPR motif residue. Instead, this observation indicates that only an A at position −2—i.e., the nucleotide present in the repeats—allows protection, and that any deviation from this nucleotide enables interference.

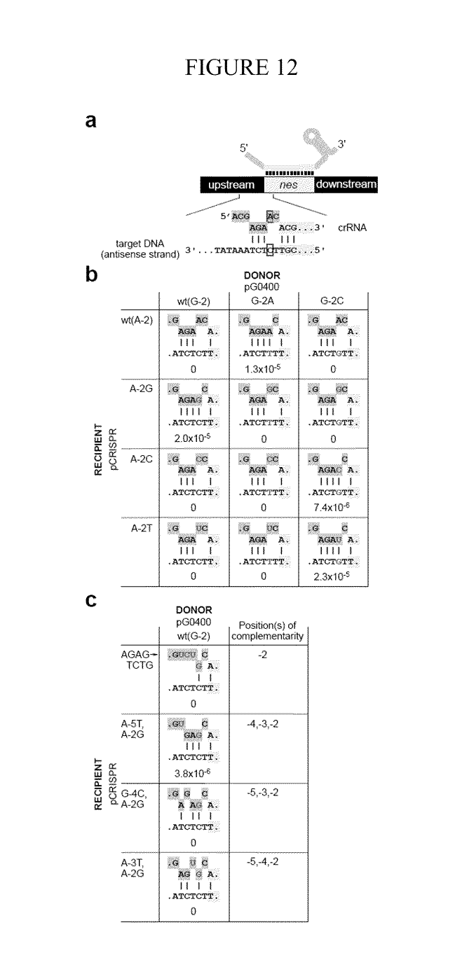

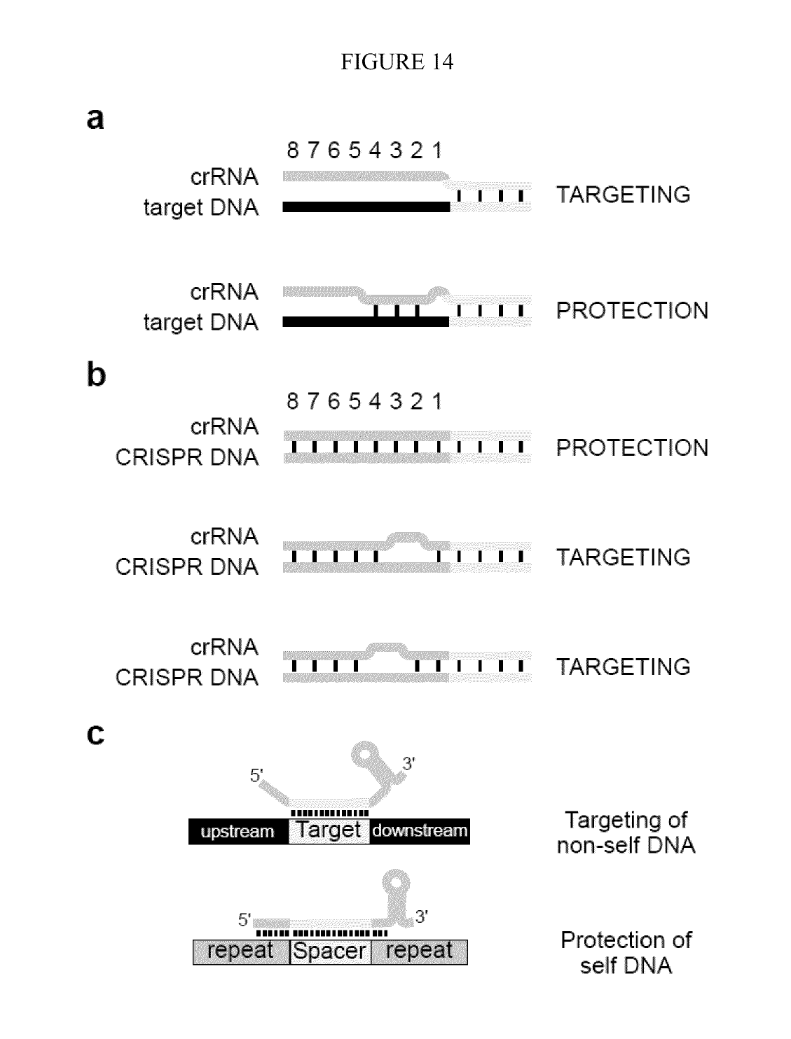

Although the spacer region of a crRNA can pair with target and CRISPR DNA alike, only the CRISPR DNA is fully complementary with the CRISPR repeat sequences at the crRNA termini (SEE FIG. 14c). In some embodiments, specific base pairs in the crRNA/DNA heteroduplex outside of the spacer region can enable protection, thereby providing a mechanism to avoid autoimmunity. Compensatory mutations were introduced in the crRNA (SEE FIG. 12a, b) by changing sequences upstream of spc1 in pCRISPR(wt), a plasmid that can complement the CRISPR interference deficiency of the LAM104 Δcrispr strain. The wild-type spc1 crRNA contains an adenosine at position −2, so we generated the A-2G, A-2C and A-2T mutants. Each mutation was tested for interference with conjugation of wild-type pG0400 as well as its G-2A and G-2C mutant derivatives. The nes target was protected from interference only when base pairing was possible at position −2: rA-dT, rG-dC, and rC-dG Watson-Crick appositions each resulted in evasion of the CRISPR system by the conjugative plasmid (SEE FIG. 12b). All crRNA mutants were functional, since all pCRISPR plasmids were able to restore CRISPR interference in LAM104 Δcrispr cells with pG0400 derivatives that were mismatched at position −2 (SEE FIG. 12b). A rU-dG wobble apposition also protected the conjugative plasmid from interference while a rG-dT wobble apposition did not, despite the greater thermodynamic stability of rG-dT pairs in an otherwise Watson-Crick-paired heteroduplex (Sugimoto et al. Biochemistry 39, 11270-11281 (2000), herein incorporated by reference in its entirety), indicating that crRNA/target noncomplementarity at position −2 is important for interference.

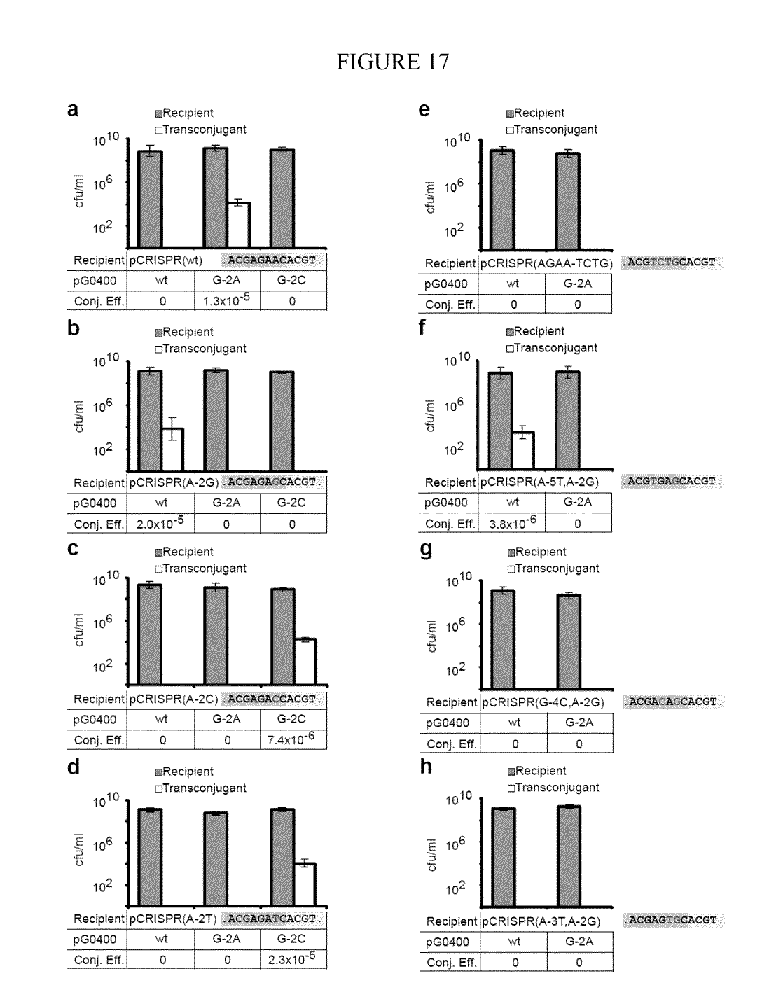

Both nes and spc1 upstream flanking sequences share the AGA trinucleotide at positions −5 to −3 (SEE FIG. 15a). Protection from interference is achieved when the potential for base-pairing between crRNA and target upstream sequences extends from position −5 to −2 (SEE FIG. 12b). Minimal complementarity required for protection was tested by introduction of additional mutations into pCRISPR(A-2G) and testing the ability of the mutant crRNAs to prevent pG0400(wt) conjugation (SEE FIG. 12c). Abolishing the complementarity at positions −5 to −3, leaving only the base pair at −2, rendered the plasmid susceptible to interference (SEE FIG. 12c), indicating that the formation of more than one base pair is required for protection. Mutation of position −5 revealed that Watson-Crick complementarity at positions −4, −3 and −2 only (pCRISPR(A-5T,A-2G), SEE FIGS. 12c and 17), confers protection from CRISPR interference. However, individual mismatches at positions −4 or −3 [pCRISPR(G-4C,A-2G) and pCRISPR(A-3T,A-2G), respectively; SEE FIG. 12c) abrogated protection. These results indicate that protection of the nes target during plasmid conjugation in S. epidermidis requires complementarity between crRNA and target upstream flanking sequences at least at positions −4, −3 and −2, and strongly suggest that the mechanism of protection requires base-pair formation in this region (SEE FIG. 14c).

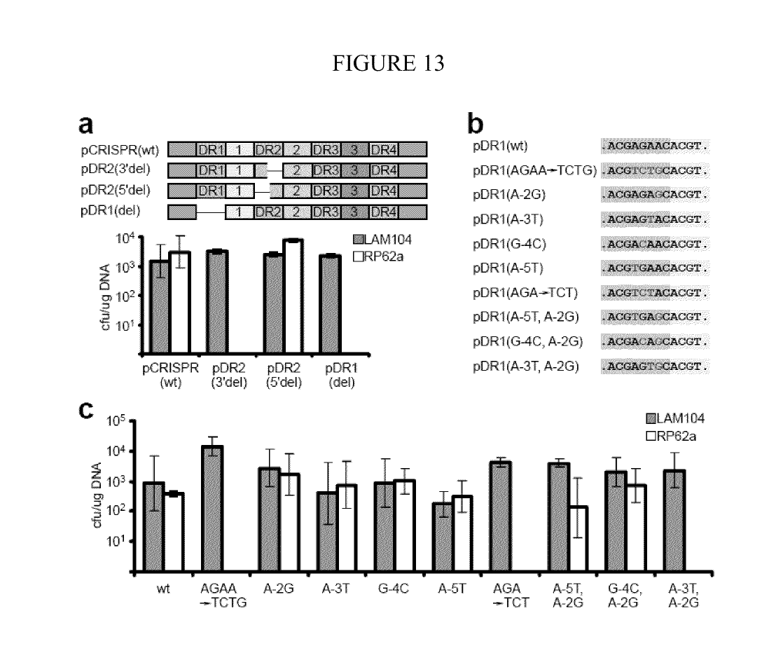

In experiments performed during development of embodiments of the present invention, it was reasoned that if base pairs at positions −4, −3 and −2 confer protection on an otherwise susceptible target, then abolition of base pairing in the same region should confer susceptibility on an otherwise protected CRISPR locus. Deletion analyses in pCRISPR (SEE FIG. 13a) demonstrated that sequences immediately upstream of spacers [spc2 in the case of pDR2(5′del) and spc1 in the case of pDR1(del)] are responsible for the prevention of autoimmunity, as these plasmids were susceptible to CRISPR interference during transformation of wild-type but not LAM104 Δcrispr cells. The effect of substitutions on the direct repeat upstream of spc1 (DR1) on CRISPR protection was analyzed (SEE FIG. 13b, c). Plasmid pDR1(AGAA→TCTG), containing mutations in the AGAA sequence at positions −5 to −2 that includes bps important for the protection of the nes target, was subject to CRISPR interference during transformation. This indicates that this region is also critical for protection of the CRISPR locus. Each of the individual mutations (A-5T, G-4C, A-3T and A-2G) failed to decrease the transformation efficiency of the respective plasmids, demonstrating that no single by is essential for protection when all other positions are complementary. Multiple mutations were introduced in this region. pDR1(AGA→TCT) as well as pDR1(A-3T,A-2G) were targeted by the CRISPR system, as they could be transformed into LAM104 Δcrispr cells but not into wild-type. In contrast, pDR1(A-5T,A-2G) and pDR1(C-4T,A-2G) resisted interference (SEE FIG. 13b, c). Other mutations outside of the AGAA sequence had no effect on protection, suggesting that at least two consecutive mismatches in the trinucleotide from positions −4 to −2 are required to eliminate protection of the CRISPR locus (SEE FIG. 14b).

Differential crRNA pairing potential with CRISPR loci and invasive targets outside of the spacer region (SEE FIG. 14c) is intrinsic to all CRISPR systems, and therefore the mechanism of self/non-self discrimination applies broadly. Experiments performed herein rationalize the previously noted 5′-terminal homogeneity of crRNAs in E. coli, S. epidermidis and P. furiosus. These crRNAs consistently contain ˜8 nt of upstream repeat sequences, in keeping with the important functional role that has been defined for this region. Results also argue against a role for the CRISPR motif during the interference phase of CRISPR immunity. Only one of the 10 by immediately upstream or downstream of the protospacers is shared between the two known targets of the S. epidermidis CRISPR locus. Furthermore, results indicate that no specific flanking nucleotides are strictly required for interference: the decisive characteristic is noncomplementarity with the crRNA rather than nucleotide identity per se.

We claim:

1. A method of inhibiting the function and/or presence of a target DNA sequence in a eukaryotic cell comprising: administering crRNA and one or more cas proteins, or nucleic acid sequences encoding said one or more cas proteins, to a eukaryotic cell comprising a target DNA sequence, wherein said crRNA hybridizes with said target DNA sequence thereby interfering with the function and/or presence of said target DNA sequence.

2. The method of claim 1, wherein said one or more cas proteins comprises Cas3.

3. The method of claim 1, wherein said one or more cas proteins comprise Cas3 and Cse1-5 proteins.

4. The method of claim 1, wherein said interfering with the function and/or presence of said target DNA sequence silences expression of said target DNA sequence.

5. The method of claim 1, wherein said cell is located in a subject.

6. The method of claim 5, wherein said target DNA sequence is a detrimental allele that causes said subject to have a disease or condition.

7. The method of claim 5, wherein said target DNA sequence is located within the genome of said cell.

8. The method of claim 7, wherein said target DNA sequence is located within close proximity to a CRISPR motif sequence.

9. The method of claim 1, further comprising studying the effect on said of interfering with the function and/or presence of said target DNA sequence compared to a control cell.

10. A method of treating or preventing an infection comprising: administering crRNA and one or more cas proteins, or nucleic acid sequences encoding said one or more cas proteins, to a subject infected by a pathogen or at risk of infection by said pathogen, wherein said crRNA hybridizes to a target DNA sequence from said pathogen thereby interfering with the function and/or presence of said target DNA sequence.

11. The method of claim 10, wherein said interfering with the function and/or presence of said target DNA sequence is fatal to said pathogen.

12. The method of claim 10, wherein said pathogen is a bacterium.

13. The method of claim 10, wherein said pathogen is a virus.

14. The method of claim 10, wherein said pathogen is a fungus.

15. A method of regulating gene transfer within a cell, tissue, or subject comprising inhibiting horizontal gene transfer, wherein clustered, regularly interspaced short palindromic repeat (CRISPR) loci and CRISPR-associated (cas) protein-coding genes are configured within the DNA of said cell, tissue, or subject to inhibit horizontal gene transfer into said DNA of said cell, tissue, or subject.

16. The method of claim 15, wherein said subject is an archeabacterium or eubacterium.

17. The method of claim 15, wherein said horizontal gene transfer comprises plasmid conjugation.

18. The method of claim 15, wherein said horizontal gene transfer comprises phage trandsduction.

19. The method of claim 15, wherein said horizontal gene transfer comprises DNA transformation.

20. The method of claim 15, wherein said CRISPR loci comprise 20-50 nucleotide direct repeats of DNA sequence separated by 24-48 nucleotide unique spacers.
